(19) United States
(12) Patent Application Publication
(10) Pub. No.: US 2019/0264228 A1
(43) Pub. Date: Aug. 29, 2019

(54) TRANSFECTION METHOD COMPRISING NONVIRAL GENE DELIVERY SYSTEMS

(71) Applicant: Rosa Karl, Munchen (DE)

(72) Inventor: Rosa Karl, Munchen (DE)

(21) Appl. No.: 16/320,113

(22) PCT Filed: Jun. 27, 2017
(86) PCT No.: PCT/DE2017/200056

§ 371 Date: Jan. 24, 2019

(30) Foreign Application Priority Data
Jul. 26, 2016 (DE) 10 2016 113 714.6

(51) Int. Cl. C12N 15/87

(52) U.S. Cl. CPC C12N 15/87

(57) ABSTRACT

A transfection method for inserting nucleic acids into eukaryotic cells using a non-viral gene delivery system is provided, the cells being treated at least with at least one inhibitor for IKKe and/or TBK1 and at least one inhibitor for nucleic-acid-detecting toll-like receptors before and/or during the transfection, and a composition and a toolkit system for a method of this type.

### BACKGROUND AND SUMMARY

The present invention relates to a method for improving the transfection efficiency of non-viral gene delivery systems and to a composition for this purpose and an associated toolkit system.

The insertion of nucleic acids (DNA, RNA etc.) into eukaryotic cells, also known as transfection, is a key technology in modem biotechnology, since it makes it possible to access the central control apparatus of a cell and thus for example to produce or switch off particular proteins. Transfection using DNA or mRNA makes it possible for example to express (produce) any desired protein; transfection using siRNA, ribozymes or antisense molecules makes it possible, for example by RNA interference, to knock down (switch off) a gene or protein. However, microRNA can also be inserted into cells, and in this way regulatory functions can be influenced. This key technology is cased in research laboratories, in medicine, and in the industrial manufacture of proteins. Not least, reference should be made to the basic application of transfection in genome editing by the CRISPR/Cas9 method, which makes it possible to alter the genome of a cell in a highly targeted manner.

In particular, this key technology makes it possible to replace genes damaged by mutation in human cells and thus to cure incorrect functionalities. Also, for example cancer cells can he forced to commit suicide using corresponding suicide genes. Knockdown of a gene is a further option for providing a curative effect, for example by shutting down important genes for angiogenesis in cancerous ulcers. Knockdown is understood to mean weakening or switching off the translation of an mRNA into a protein in protein biosynthesis. Many diseases are triggered by incorrect control of cells, which could be eliminated using microRNA.

The success of transfection of this type is defined among other things using transfection efficiency. Transfection efficiency is understood to be the level of a protein expression of a cell population as a result of transfection processes using genetic material which codes among other things this expressed protein, or else the extent of a knockdown of a protein expression of a cell population as a result of transfection process using genetic material which can trigger a knockdown of this type. In particular siRNA, antisense RNA, ribozyme, antisense DNA, or DNA coding for siRNA or ribozyme may be used as genetic material. Often, the transfection efficiency is also defined by the proportion of cells, within a total population of cells, which exhibit biological effectiveness of the inserted genetic material as a result of transfection processes.

So as to be able to insert nucleic acids into cells, gene delivery systems are generally required which at least make the membrane barrier of the cells permeable to the nucleic acid. In the following, transfection therefore understood to mean treatment of eukaryotic cells using a gene delivery system and nucleic acids.

Usually, the gene delivery systems present are subdivided into two main groups, namely viral systems and non-viral systems.

In viral systems, which are preferred in particular in gene therapy research, viruses are used as gene delivery systems. Since the introduction of nucleic acids, in particular DNA or RNA, into foreign cells is an integral part of the reproduction cycle of viruses, this ability has been refined through a natural evolutionary process in the developmental history of viruses to such an extent that if forms an extremely effective gene delivery system. The viruses used are altered by gene manipulation in such a way that they no longer have any pathogenic properties and can no longer reproduce.

However, a drawback of viral systems is that the viruses provide an easy target for the immune system, since the immune system has developed strategies to combat viruses in a likewise evolutionary adaptation process. Immune defence and the activation of oncogenes by random integration of genetic material into the genome are unsolved problems, and so in spite of decades of research worldwide there are only isolated permitted gene therapies. In basic research, although viruses are also often used, because of the safety risks and the complex handling the viral systems are only used when there is basically no alternative.

In non-viral gene delivery systems, generally no naturally occurring viruses are used, and they are not generated by recombination of genetic materials of naturally occurring viruses. These non-viral systems may in turn be subdivided into two sub-groups, systems based on chemical methods and systems based on physical methods.

The non-viral gene delivery systems based on chemical methods either involve chemical alteration to or derivatisation of the nucleic acids themselves, which make them able to permeate cells, or comprise substances which bind nucleic acids, for example using electrostatic forces or hydrogen bridge bonds, and can induce transport through the cell membrane. The transport of the nucleic acid through the cell membrane generally takes place using an active transport mechanism of the cell known as endocytosis.

Substances which make binding of the nucleic acids possible include for example cationic lipids, cationic polymers, cationic peptides. In the presence of DNA or RNA, as a result of the opposed charge relationships, these cationic lipids and cationic polymers spontaneously form lipoplexes or polyplexes. The DNA is condensed, in other words minimised in size, by the compensation of the negative charge on the phosphate group. These complexes can be absorbed by the cells by endocytosis. Mixed forms are also sometimes used, in which the nucleic acids are precondensed by cationic polymers and subsequently complexed by cationic lipids into a mixed form consisting of lipoplexes and polyplexes. Frequently, for example polylysine, polyarginine or polyethylenimine are used for this purpose. Naturally, other cationic polymers and peptides known to a person skilled in the an ma also be used.

However, the substances suitable for a chemical-basis non-viral gene delivery method may also be molecules having at least a first and a second domain/molecule part. The first domain is formed as a nucleic-acid-binding domain/molecule part. A nucleic-acid-binding molecule part is understood to be a region in a molecule which binds a nucleic acid, in particular DNA and/or RNA, covalently or by way of non-covalent interactions, in particular electrostatic forces and hydrogen bridge bonds. The second domain/molecule part preferably includes a ligand. This ligand may for example be recognised by a receptor on the cell surface and trigger endocytosis as a result of this recognition process.

Alternatively, this ligand may be capable of triggering a membrane transfer, in other words inducing transport to the other side of the membrane. Membrane transfer is understood to mean that a molecule can pass from one side of the membrane to the other side. However, the ligands which can trigger a receptor-induced endocytosis or a membrane transfer may also be covalently bonded to the genetic material if the biological effect is not or is only slightly impaired as a result.

The substances may also be specially formulated, in particular as micelles or liposomes, or else comprise a plurality of components having different functionalities.

Non-viral gene delivery systems based on physical methods localise the genetic material close to the cell and use energy in particular in the form of thermal, kinetic, electrical or other energy so as to induce transport of the genetic material through the cell membrane. Electroporation should be mentioned as an important example of a non-viral method based on a physical method. In this method, the cells to be transfected are passed between two electrodes, to which a suitable voltage progression is applied. In this way, the cells are exposed to an electrical pulse, which brings about reversible opening (pores) of the cell membrane. Nucleic acids can penetrate into the cell through these pores.

Further important physical methods are microinjection, hydrodynamic methods, ballistic methods (gene guns) or methods using ultrasound. These also include methods in which the nucleic acid is injected bare into different organs or muscles, potentially leading to lower expression of the acne in question in special cases.

Combined methods such as magnetofection combine chemical and physical methods. In this context, nucleic acids are chemically in on magnetic nanoparticles so as to accumulate them on the surface of cells using a magnetic field gradient and trigger endocytosis.

However, a drawback of all non-viral systems is that, as noted above, the efficiency thereof does not reach that of viral systems. However, since the viral method can only be used in a very restricted manner as a result of the various drawbacks, correspondingly high-performance alternatives are being sought among non-viral methods.

In the prior art, for example WO 2009/065618 or WO2010/133369, it has already been found that the innate immune system can form a barrier to successful transfection, since the cells are capable of detecting nucleic acids using endosomal and cytosolic receptors and thereupon changing their physiological properties with the aim of defending against microbial attack. It has therefore been proposed in the prior art to act on the immune system in a targeted manner and thus to increase the transfection efficiency.

However, a problem with this approach is that in large regions the innate immune system has a redundant construction, which comprises an enormous number of interacting mechanisms, some of which have not yet been researched and whose relationships have not yet been determined. The redundancy stems from the evolutionary battle between bacteria and viruses on the one hand and eukaryotes on the other hand. If a signal thread is to be interrupted by a pathogen as an attack strategy, because of the redundant construction of the innate immune system the cell is not left defenceless. As a result, the interruption of a signal thread generally only provides moderate increases in transfection efficiency.

It is desirable, therefore, on the basis of an immune system suppression mechanism, to provide a transfection method which is based on a non-viral gene delivery system and which makes it possible to increase the transfection efficiency.

This object is achieved by a method according to an aspect of the invention, a composition according to an aspect of the invention and a toolkit system according to an aspect of the invention.

The invention, according to an aspect thereof, proposes a transfection method for inserting one or more nucleic acids into eukaryotic cells using a non-viral gene delivery system, in which the performance of the non-viral gene delivery system is improved in that the cells are treated at least with at least one inhibitor for IKKe and/or TBK1 and at least one inhibitor for at least one nucleic-acid-detecting toll-like receptor before and/or during the transfection.

A toll-like receptor (TLR for short) is understood to refer to proteins of the innate immune system. They belong to a group of receptors which are for recognising pathogenic structures and control corresponding gene activations. As a result, in particular the activation of the antigen-specific acquired immune system is initiated and modulated. By way of the toll-like receptors, the innate immune system can distinguish between “self” and “non-self”. More precisely, the TLRs are transmembrane proteins having an extracellular leucine-rich repeat (LRR) domain and a cytoplasmic domain, which is homologous with that of the IL-IR family. The various TLRs react selectively to different molecular viral and bacterial components, and control a corresponding activation of genes via a signal transduction cascade. This initially takes place by way of what are known as adapter molecules, and subsequently by way of kinases, which ultimately activate transcription factors (for example NF-κB and the IRF families), by phosphorylating them, or corresponding intracellular inhibitors of these transcription factors. Finally, alongside a large number of specific genes which have an antimicrobial effect, cytokines are produced. Cytokines are also necessary stimulators for the acquired immune system and thus also form a link between the innate and the acquired immune system.

Thus far, 13 different TLRs are known (of which 10 in humans), of which in turn only three are currently classified as nucleic-acid-detecting in humans: TLR3 (long dsRNA), TLR7 (ssRNA/dsRNA for example of RNA viruses) and TLR9 (bacterial/viral DNA).

IKKe and TBK1 are kinases which play a major role in the signal transduction cascade in the innate immune system, which is downstream from a large number of cytosolic receptors and ends in the activation of the transcription factors IRF3 and IRF7. The kinase IKKe is also referred to as IKKε, IκKε, Iκ kinase ε or IKK3. TBK1 is also referred to as TANK-binding kinase 1. Since IKKe and TBK1 are two closely related kinases, inhibitors against IKKe generally also act against TBK1 and vice versa.

According to an aspect of the invention, an inhibitor is understood to mean a molecule which can reduce or inhibit the biological effect of another molecule, in particular a protein. The inhibitors, are themselves proteins, modified or unmodified nucleic acids or small organic molecules, it also being possible for suitable siRNAs which suppress the expression of a protein to be counted as inhibitors. In this case, the siRNA has tau be inserted using the gene delivery system if applicable. The inhibitive effect may also come about as a result of a molecule being masked which normally is recognised by a protein and thus triggers a biological effect. The effectiveness of an inhibitor is specified using the IC₅₀ or EC₅₀. The IC₅₀ specifies the concentration of an in which is necessary for 50% blocking of a target for example enzyme, cell, cell receptor, microorganism etc.) in vitro. The EC₅₀, the effective concentration, specifies this necessary concentration in vivo.

The IKKe/TBK1 kinase and the TLRs are both known as influencing factors for the effectiveness of the immune system. However, entirely unexpectedly, it has been found that cells which are treated with a combination of an inhibitor for the kinase IKKe and/or the kinase TBK1 and an inhibitor for nucleic-acid-detecting toll-like receptors, in other words in which the effect of IKKe/TBK1 kinases and TLRs is switched off, exhibit a synergistic increase in transfection efficiency when non-viral gene delivery systems are used. The increase due to the combination is greater than the sum of the increases for the individual components.

In a further advantageous embodiment, the transfection efficiency of this method is increased in particular in that an inhibitor for toll-like receptor 9 (TLR9) is used. TLR9 is a receptor for bacterial DNA, or for non-methylated CpG motifs, which occurs frequently in bacterial DNA (20 times more frequently than in mammalian cells). The CpG motif is heavily methylated in mammalian cells, meaning that it can be distinguished. The situation as regards bacterial DNA similarly applies to viral DNA, which is also detected by TLR9.

In a further embodiment, as an inhibitor for IKKe/TBK1, an inhibitor is used having an IC₅₀ of less than 500 nM, preferably less than 200 nM, most preferably less than 100 nM, and/or, as an inhibitor for the nucleic-acid-detecting toll-like receptor, an inhibitor is used having an EC₅₀ value of less than 1000 nM, preferably less than 500 nM, most preferably less than 200 nM, Inhibitors having an IC₅₀ or EC₅₀ of this type make possible particularly good inhibition, which significantly increases the transfection efficiency.

In a further advantageous embodiment, as an inhibitor for the nucleic-acid-detecting toll-like receptor, a compound from the group of the 9-aminoacridines and/or the 4-aminoquinolines, including the salts thereof, is used.

The group of the 4-aminoquines includes compounds and the salts thereof having the following basic structure, where R1 to R7 may be any desired substituents.

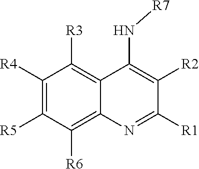

The group of the 9-aminoacridines includes compounds and the salts thereof having the following basic structure, where R1 to R4 may again be any desired substituents.

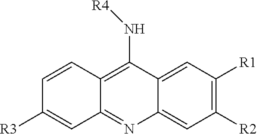

In a preferred embodiment, as an inhibitor for at least one toll-like receptor, quinacrine from the group of the 9-aminoacridines and/or chloroquine from the group of the 4-aminoquinolines may be used.

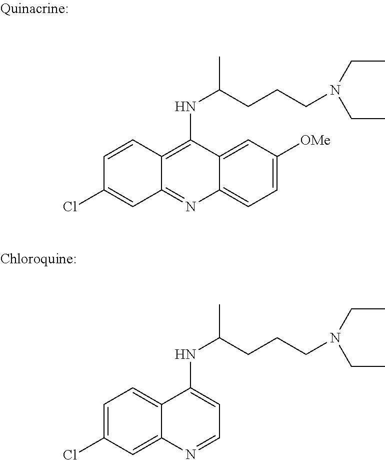 [SMILES: CCN(CC)CCCC(C)Nc1c2ccc(Cl)cc2nc2ccc(OC)cc12 ; CCN(CC)CCCC(C)Nc1ccnc2cc(Cl)ccc12]

Although chloroquine has already previously been used for increasing the transfection efficiency in transfection processes, but exhibit highly inconsistent properties in the sole use analysed thus far, in such a way that the transfection efficiency could not be increased reliably. Only by way of the combination according to an aspect of the invention with an inhibitor of IKKe/TBK1 kinase could a reliable and significant increase in the transfection efficiency be achieved.

In a further preferred embodiment, the following compounds from the group of the 9-aminoacridines and 4-aminoquinolines are used as inhibitors:

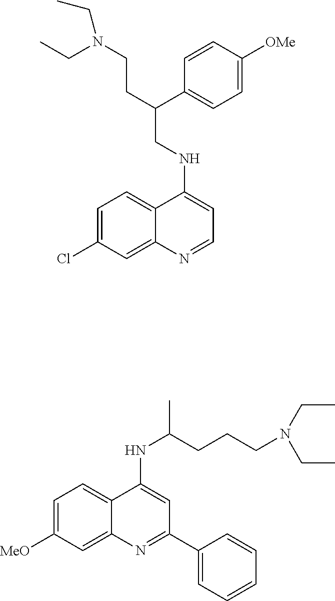 [SMILES: CCN(CC)CCC(CNc1ccnc2cc(Cl)ccc12)c1ccc(OC)cc1 ; CCN(CC)CCCC(C)Nc1cc(-c2ccccc2)nc2cc(OC)ccc12]

These compounds too exhibit a considerable increase in transfection efficiency in combination with an inhibitor of IKKe/TBK1 kinase.

Alternatively, as an inhibitor for a toll-like receptor, an oligonucleotide of which the sequence is suitable for inhibiting toll-like receptors may also be used, or antibodies directed against toll-like receptors may also be used. Inhibition may naturally also be achieved using a combination of the aforementioned embodiments.

In this case too, further inhibitors for nucleic-acid-detecting toll-like receptors which are only subsequently recognised as such may also be included in the scope of an aspect of the invention. Also, in addition to the combination of an inhibitor for IKKe/TBK1 and an inhibitor for a toll-like receptor, further inhibitors may also be used.

In a particularly preferred embodiment of an aspect of the invention, as an inhibitor for IKKe/TBK1, one or more of the following inhibitors, including the salts thereof, are used;
BX795 (N-(3-((5-iodo-4-((3-(2-thienylcarbonyl) amino) propyl) amino)-2-pyrimidinyl) amino) phenyl)-1-pyrrolidinecarboxamide, CAS 702675-74-9);
BX320 (N-(3-((5-bromo-2-(3-(pyrrolidine-1-carbonylamino) anilino) pyrimidin-4-yl)amino) propyl)-2,2-dimethyl propanediamide, CAS 702676-93-5);
Cay10576 (5-(5,6-dimetboxybenzimidazol-1-yl)-3-(2-methanesulphonyl-benzyloxy)-thiophene-2-carbonitrile, CAS 862812-98-4);
Cay10575 (5-(5,6-dimethoxybenzimidazol-1-yl)-3-((4-methylsulphonyl) phenyl) methoxy)-2-thiophenecarboxamide, CAS 916985-21-2);
Amlexanox (2-amino-7-(-methylethyl)-5-oxo-5H-[1]Benzopyrano[2,3-b]pyridine-3-carboxylic acid, CAS 68302-57-8);
MRT-67307 (N-[3-[[5-cyclopropyl-2-[[3-(4-morpholinylmethyl)phenyl]amino]-4-pyrimidinyl]amino]propyl]-cyclobutanecarboxamide, CAS 1190378-57-4);
CYT387 (N-(cyanomethyl)-4-[2-[[4-(4-morpholinyl)phenyl]amino]-4-pyrimidinyl]-benzamide, CAS 1056634-68-4).

With the advantageous use of these inhibitors, an increase in transfection efficiency can clearly be seen.

As further known inhibitors for IKKe/TBK1, SU6668 (Sugen Inc.), MPI-0485520 (Myriad Pharma), MCCK1 and the Amgen TBK 1 inhibitor (Compound II) (Ou et al.; Molecular Cell; 2011; 41; 458-470) may be used. EP 1720864, WO 2009030890, WO 2010100431, WO 2012059171, WO002012161877, WO002012161879, WO002013034238, WO002013024282, WO002013075785, WO002013117285, WO002014189806, WO002014128486 WO002014093936, WO002015134171, WO002016057338, US020150352108and US020160015709 cite further IKKe/TBK1 inhibitors which may also be used.

Other inhibitors not cited above may also be used if they have an inhibitive effect on IKKe and/or TBK1. Inhibitors of which the effect on IKKe and/or TBK1 is only recognised at a later time are also included.

In a further advantageous embodiment of the method according to an aspect of the invention, the nucleic acid inserted by transfection is in particular modified and/or unmodified ssDNA, modified and/or unmodified dsDNA, modifed and/or unmodified ssRNA, modified and/or unmodified dsRNA. In this context, dsDNA and ssRNA have been found to be particularly preferred. Various types of nucleic acids may also be used in combination, as is often required in genome editing by the CRISPR/Cas9 method.

A nucleic acid is understood to mean a ribonucleic acid or a deoxyribonucleic acid which consists in particular of two at least partially complementary strands (double-strand=ds), for example dsDNA and dsRNA, or which consists of one strand (single-strand=ss), for example ssDNA and ssRNA, which may have complementary regions in part. In the case of DNA, the genetic material is for generating RNA and/or proteins. In the case of ssRNA, it is for generating proteins. In the case of dsRNA, the genetic material serves to achieve knockdown of a gene by RNA interference or to act as microRNA. As antisense DNA or antisense RNA, the nucleic acid serves to inhibit the translation of mRNA.

Modified nucleic acid refers to natural nucleic acids of which the properties have been altered by modification. These modifications may in particular be chemical alterations which affect the phosphate backbone and/or the sugars and/or the bases, this being intended in particular to increase the stability of the nucleic acids against nucleases and ribonucleases and to decrease their recognisability for nucleic-acid-detecting receptors.

Furthermore, molecules (labels) which lead to new properties of the nucleic acids, in particular to optical traceability using fluorescence labels, or labels which direct the nucleic acids to a particular location in the cell (localisation elements), or labels which induce passage of nucleic acids through membranes and thus make nucleic acids for example capable of accessing cells, may be covalently or non-covalently attached to the nucleic acids. Examples of modifications are the replacement of oxygen with sulphur in the phosphate backbone, in the case of RNA the methylation of 2′ OH groups of the ribose, or the methylation of bases. A further example is complete substitution of 2′ OH groups of RNA with fluorine to increase stability against nucleases. Another further example is the attachment of FITC (fluorescein isothiocyanate) as a fluorescence label so as to be able to trace the path of the genetic material in the cell or the attachment of NLSs (nuclear localisation signals, for example PKKKRKVG) so as to achieve transport into the cell nucleus.

As a non-viral gene delivery system, any gene delivery system known to a person skilled in the art may be selected. In particular, as a further embodiment shows, it is preferred if, as a non-viral gene delivery system, a gene delivery system is used which:
comprises a cationic lipid, a cationic polymer or a cationic protein; and/or
comprises a compound which include a DNA-binding and/or RNA-binding domain and which can trigger a receptor-induced endocytosis or a membrane transfer; and/or
comprises a compound which is covalently bonded to DNA and/or RNA and which can trigger a receptor-induced endocytosis or a membrane transfer.

Alternatively or in addition, the non-viral gene delivery system may also be based on a physical method such as electroporation, microinjection, magnetofection, ultrasound or a ballistic or hydrodynamic method.

A further aspect of the present invention further provides a composition of at least one inhibitor for IKKe and/or TBK1 and at least one inhibitor for at least one nucleic-acid-detecting toll-like receptor. Preferably, the composition may further comprise at least one non-viral gene delivery system and/or one or more modified or unmodified nucleic acids.

Preferably, one or more of the above-discussed inhibitors, gene delivery systems and/or nucleic acids may be comprised in the composition.

A further aspect of the present invention further provides a toolkit system for carrying out the above-discussed transfection method comprising at least one first inhibitor sub-composition, which comprises at least one of the above-discussed inhibitors for IKKe and/or TBK1, and a second inhibitor sub-composition, which comprises at least one of the above-discussed inhibitors for at least one nucleic-acid-detecting toll-like receptor.

Alternatively, in this aspect of the invention, the toolkit system may also provide an inhibitor composition which comprises at least one inhibitor for IKKe and/or TBK1 and simultaneously at least one inhibitor for at least one nucleic-acid-detecting toll-like receptor.

In addition, the toolkit system may provide a gene delivery composition, comprising at least one non-viral gene delivery system, and/or a nucleic acid composition, comprising at least one modified or unmodified nucleic acid.

The inhibitor composition or at least one of the inhibitor sub-compositions is a composition of inhibitors, as discussed above.

In a further embodiment, in the toolkit system, one or more of the compositions or sub-compositions may form a combined composition together with one or more other compositions or sub-compositions.

This means that in the toolkit system according to an aspect of the invention, all of the components may be present separately from one another or be present together in all combinatorically possible combinations as a composition. Thus, the components may either be present separately from one another, for example in class or plastics containers which are packaged together, or two or more of the compositions may be provided in corresponding containers as a composition.

The composition according to an aspect of the invention and/or the toolkit system according to an aspect of the invention can be used for carrying out the method according to an aspect of the invention. Further, the composition according to an aspect of the invention may be present as a pharmaceutical composition. Further, the composition according to an aspect of the invention or a toolkit system may be used for treating a disease by gene therapy, for “genome editing” for example by CRISPR-Cas9, or else for repeated transfection of cells.

Further advantages and advantageous embodiments are defined in the claims, description and drawings. Further, the described features may be present individually or in combination. In addition, unless stated otherwise, features which are described in combination may be present as individual features or in combinations other than the stated combination.

### BRIEF DESCRIPTION OF THE DRAWINGS

In the following, an aspect of the invention into be described in greater detail by way of embodiments shown in the drawings. The embodiments are purely exemplary in nature and are not intended to define the scope of protection of the application, which is defined solely by the accompanying claims.

In the drawings:

FIG. 1: is a graphical representation of transfection efficiencies in accordance with a first embodiment; and

FIG. 2: is a graphical representation of transfection efficiencies in accordance with a second embodiment.

### DETAILED DESCRIPTION

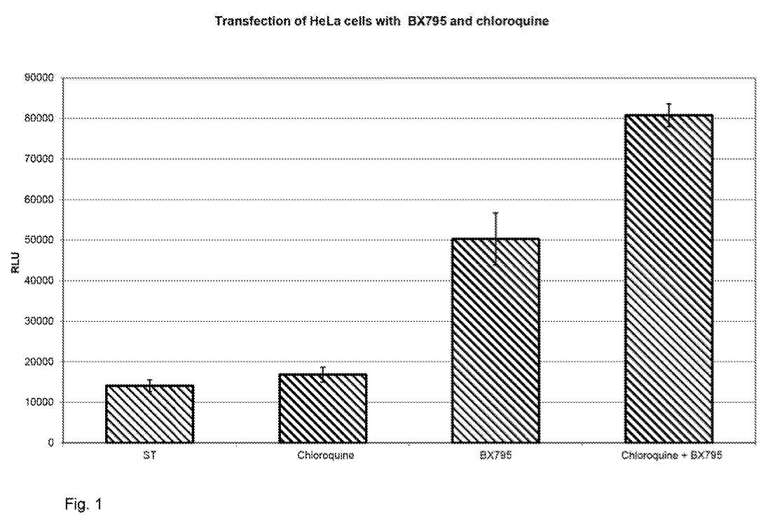

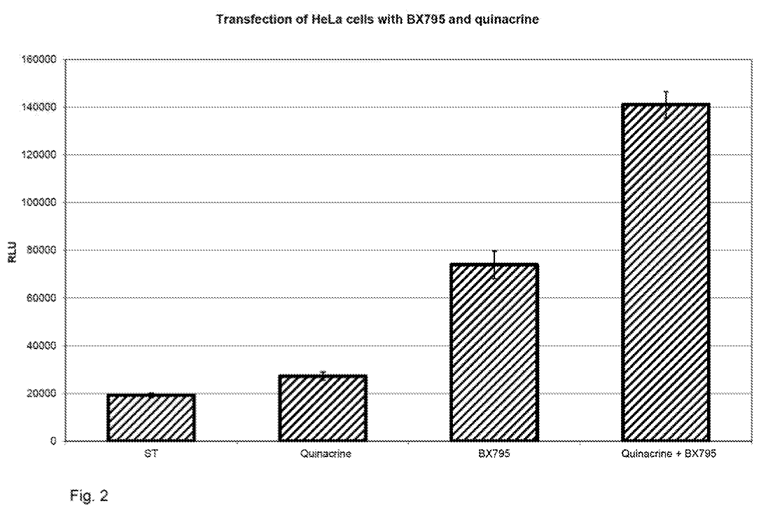

FIG. 1 and FIG. 2 are schematic comparative representations of transfection efficiencies achieved with the method according to an aspect of the invention and without the method according to an aspect of the invention.

As can be seen from the drawings, using the treatment according to an aspect of the invention of the cells before and/or during the transfection a considerable increase in the transfection efficiency can be achieved. The transfection efficiency was measured indirectly using a luciferase enzyme, which was coded by the inserted nucleic acid. This is a reporter gene system. These are established systems for detecting the transfection efficiency. Thus, the greater the amount of luciferase which can be detected in a culture vessel after lysis of the transfected cells, the greater the transfection efficiency. The amount of luciferase is detected by way of an enzyme substrate reaction in which a light quantum is released. These light quanta can be measured by suitable measurement devices known as luminometers. Since the number of light quanta measured is dependent in particular on the time interval in which the measurement took place, this light quantum amount is also denoted as “relative light units”. For comparative studies, the measurement conditions must therefore be identical.

As can be seen from the drawings, the different treatments of the cells are plotted on the x-axis and the transfection efficiency in [%] achieved for each of the treatments is plotted on the y-axis. The measurement values have been normalised to transfection without inhibitors by setting this equal to 100%. In both examples, for the treatment using the IKKe/TBK1 inhibitor and an inhibitor for a nucleic-acid-detecting toll-like receptor a significant improvement can be observed, and also cannot be explained merely by simple addition of the individual rates of increase for TKKe/TBK1 inhibitor and an inhibitor for a nucleic-acid-detecting toll-like receptor. This is thus clearly a synergistic effect.

As regards the drawings, in detail:

The following was used as general material:
1. HeLa cells; ATTC CCL-2
2. Rotifect Plus, Carl Roth; Cat. No.: CL21.2
3. 48-well plates; Cellstar; Greiner BioOne; Cat. No.: 677180
4. DMEM; high-glucose; Biowest, w/stable glutamine/sodium pyruvate, Cat. No.: L103-500
5. Serum FBS Gold; PAN Biotech; Batch No. P130914: Cat. No.: P40-37500
6. pCMV-luc, Plasmid Factory, Cat. No.: PF461
7. Luciferase Assay Kit, Promega
8. Dimethyl sulphoxide (DMSO for molecular biology), Fluka; Cat. No.: 41639
9. (N-(3-((5-Iodo-4-((3-(2-thienylcarbonyl)amino)propyl)amino)-2-pyrimidinyl)amino) phenyl)-1-pyrrolidinecarboxamide (BX795), Merck4Biosciences, Cat. No.: 204001
10. Chloroquine diphosphate; Fluka; Cat. No.: C6628
11. Hydrated quinacrine dichloride; TCI: Cat. No.: Q0056

### EXAMPLE 1

### FIG. 1

The first embodiment, shown in FIG. 1, relates to lipofection of HeLa cells with a conventional commercial transfection reagent (Rotifect Plus), which were treated with the inhibitor BX795 for IKKe and TBK1 and with chloroquine as an inhibitor for TLR9 before and during the transfection.

The lipofection of the HeLa cells was carried out over a 3-day period:

1^{st} day: HeLa cells were seeded into a 48-well plate. A cell count of 1*10⁵ cells per well was plated in 250 μl complete DMEM medium (10% FCS). This was followed by incubation in a CO₂ incubator (10%) for 24 hours.

2^{nd} day:

From chloroquine and BX795, stock solutions were produced in a mixture of DMSO and water. 2 hours before the transfection, 4 wells in each case were filled with chloroquine or BX795 and 4 wells are filled with both substances. Using a corresponding amount of the two stock solutions, the concentration of the inhibitors in the culture medium of the cells is set in such a way that chloroquine was present at a concentration of 10 μM and BX795 was present at a concentration of 0.5 μM. It was further ensured that the DMSO concentration did not exceed 1% v/v. The transfection of all of the cells in the 48-well plate was carried out using 0.3 μg pCMV-Luc and 1.2 μl Rotifect Plus, in accordance with the manufacturer's specifications for the transfection reagent. This was followed by incubation in a CO₂ incubator (10%) for 24 hours.

3^{rd} day:

The efficiency of the transfection was determined using a luciferase assay kit. The assay was carried out in accordance with the manufacturer's instructions.

Results in RLU (averages of three tests)

| | | | BX795 + |
|---|---|---|---|
| ST | Chloroquine | BX795 | chloroquine |
| 14087 | 46817 | 50282 | 80788 |
| 100% | 119% | 356% | 574% |

ST = Standard transfection without BX795 and chloroquine
BX795: C = 0.5 μM
Chloroquine: C = 10 μM

The increases over standard transfection resulting from individual application of chloroquine and BX795 are 19% and 256% respectively. At 474%, the increase resulting from combined application is far more than the total of the increases in the case of individual application; in other words, the combined application exhibits a synergistic effect.

### EXAMPLE 2

### FIG. 2

The second embodiment, shown in FIG. 2, relates to lipofection of HeLa cells, which were treated with the inhibitor BX795 for IKKe and TBK1 and with quinacrine as an inhibitor for TLR9 before and during the transfection.

The lipofection of the HeLa was carried out analogously with Example 1.

Results in RLU/μg protein (averages)

| ST | BX795 | Quinacrine | BX795 + quinacrine |
|---|---|---|---|
| 19271 | 27260 | 73924 | 141056 |
| 100% | 141% | 385% | 732% |

ST = Standard transfection without BX795 and quinacrine
BX795: C = 0.5 μM
Quinacrine: C = 2.5 μM

The increases over standard transfection resulting from the individual application of quinacrine and BX795 are 41% and 285% respectively. At 632%, the increase resulting from combined application is far more than the total of the increases in the case of individual application; in other words, the combined application exhibits a synergistic effect.

Generally speaking, it can thus be concluded that, advantageously, as a result of the treatment according to an aspect of the invention of the cells before and/or during the transfection at least with at least one inhibitor for IKKe and/or TBK1 and at least one inhibitor for at least one nucleic-acid-detecting toll-like receptor, a significant increase in transfection efficiency involving a synergistic effect can be observed.

1. Transfection method for inserting one or more nucleic acids into eukaryotic cells using a non-viral gene delivery system,
wherein
the cells are treated at least with at least one inhibitor for IKKe and or TBK1 and at least one inhibitor for at least one nucleic-acid-detecting toll-like receptor, TLR, before and/or during the transfection.

2. Transfection method according to claim 1, wherein, as an inhibitor for the nucleic-acid-detecting toll-like receptor, at least one inhibitor for TLR9, toll-like receptor 9, is used.

3. Transfection method according to claim 1, wherein, as an inhibitor for IKKe/TBK1, an inhibitor is used having an IC₅₀ of less than 500 nM, preferably less than 200 nM, most preferably less than 100 nM; and/or,
as an inhibitor for the nucleic-arid-detecting toll-like receptor, an inhibitor is used having an EC₅₀ of less than 1000 nM, preferably less than 500 nM, most preferably less than 200 nM.

4. Transfection method according to claim 1, wherein, as an inhibitor for the nucleic-acid-detecting toll-like receptor, at least one compound from the group of the 4-aminoquinolines, including the salts thereof, is used, the compound having the following basic structure: 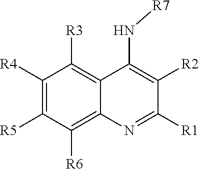
where R1 to R7 may be selected as desired.

5. Transfection method according to claim 4, wherein, as an inhibitor for the nucleic-acid-detecting toll-like receptor, at least chloroquine, including the salts thereof, is used, with R5=Cl and R7=NHCHMe(CH₂)₃NEt₂ and according to the following structure: 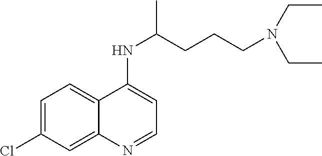 [SMILES: CCN(CC)CCCC(C)Nc1ccnc2cc(Cl)ccc12]
and/or, as an inhibitor for the nucleic-acid-detecting toll-like receptor, at least one compound, including the salts thereof, is used, with R5Cl and R7=NHCH₂CH(C₆H₄OMe)(CH₂)₂NEt₂ and according to the following structure: 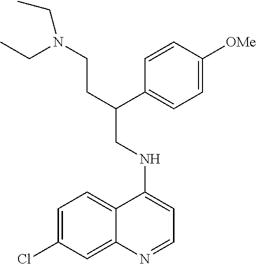 [SMILES: CCN(CC)CCC(CNc1ccnc2cc(Cl)ccc12)c1ccc(OC)cc1]
and/or, as an inhibitor for the nucleic-acid-detecting toll-like receptor, at least one compound, including the salts thereof, is used, with R5=OMe, R7=NHCHMe(CH₂)₃NEt₂, R1=C₆H₅ and according to the following structure:

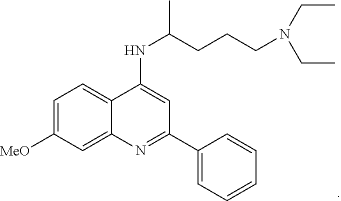 [SMILES: CCN(CC)CCCC(C)Nc1cc(-c2ccccc2)nc2cc(OC)ccc12]

6. Transfection method according to claim 1, wherein, as an inhibitor for the nucleic-acid-detecting toll-like receptor, at least one compound from the group of the 9-aminoacridines, including the salts thereof, is used, the compound having the following basic structure: 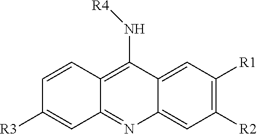
where R1 to R4 may be selected as desired.

7. Transfection method according to claim 6, wherein, as an inhibitor for the nucleic-acid-detecting toll-like receptor, at least quinacrine, including the salts thereof, is used, with R1=OMe, R2=H, R3=Cl, R4=NHCHMe(CH₂)₃NEt₂ and according to the following structure:

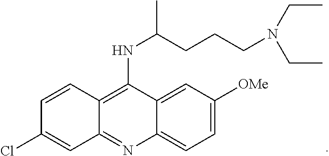 [SMILES: CCN(CC)CCCC(C)Nc1c2ccc(Cl)cc2nc2ccc(OC)cc12]

8. Transfection method according to claim 7, wherein, as an inhibitor for IKKe and/or TBK1, one or more of the group of
BX795 (N-(3-((5-iodo-4-((3-(2-thienylcarbonyl) amino) propyl) amino)-2-pyrimidinyl) amino) phenyl)-1-pyrrolidinecarboxamide, CAS 702675-74-9);
BX320 (N-(3-((5-bromo-2-(3-(pyrrolidine-1-carbonylamino) anilino) pyrimidin-4-yl)amino) propyl)-2,2-dimethyl propanediamide, CAS 702676-93-5);
Cay10576 (5-(5,6-dimethoxybenzimidazol-1-yl)-3-(2-methanesulphonyl-benzyloxy)-thiophene-2-carbonitrile, CAS 862812-98-4);
Cay10575 (5-(5,6-dimethoxybenzimidazol-1-yl)-3-((4-methylsulphonyl) phenyl) methoxy)-2-thiophenecarboxamide, CAS 916985-21-2);
Amlexanox (2-amino-7-(1-methylethyl)-5-oxo-5H-[1]benzopyrano[2,3-b]pyridine-3-carboxylic acid, CAS 68302-57-8);
MRT-67307 (N-[3-[[5-cyclopropyl-2-[[3-(4-morpholinylmethyl)phenyl]amino]-4-pyrimidinyl]amino]propyl]-cyclobutanecarboxamide, CAS 1190378-57-4);
CYT387 (N-(cyanomethyl)-4-[2-[[4-(4-morpholinyl)phenyl]amino]-4-pyrimidinyl]-benzamide, CAS 1056634-68-4);
including the salts thereof, are selected.

9. Composition for a transfection method according to claim 1 wherein the composition comprises at least one inhibitor for IKKe and/or TBK1 and at least one inhibitor for at least one nucleic-acid-detecting toll-like receptor.

10. Composition according to claim 9, wherein the composition further comprises:
a non-viral gene delivery system, and/or
one or more modified or unmodified nucleic acids.

11. Composition according to claim 9, wherein the at least one inhibitor for the nucleic-acid-detecting toll-like receptor is an inhibitor for TLR9, toll-like receptor 9.

12. Composition according to claim 9, wherein the inhibitor for IKKe/TBK1 has an IC₅₀ of less than 500 nM, preferably less than 200 nM, most preferably less than 100 nM; and/or
the inhibitor for the nucleic-acid-detecting toll-like receptor has an EC₅₀ of less than 1000 nM, preferably less than 500 nM, most preferably less than 200 nM.

13. Composition according to claim 9, wherein the inhibitor for the nucleic-acid-detecting toll-like receptor comprises at least one compound from the group of the 4-aminoquinolines, including the salts thereof, which has the following base structure: 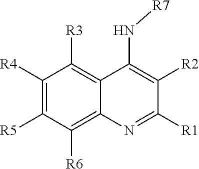
where R1 to R7 may be selected as desired.

14. Composition according to claim 13, wherein the inhibitor for the nucleic-acid-detecting toll-like receptor comprises at least chloroquine, including the salts thereof, with R5=Cl and R7=NHCHMe(CH)₃NEt₂ and according to the following structure: 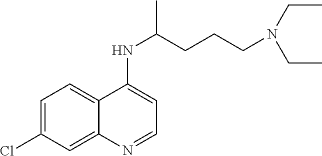 [SMILES: CCN(CC)CCCC(C)Nc1ccnc2cc(Cl)ccc12]
and/or the inhibitor for the nucleic-add-detecting toll-like receptor comprises at least one compound, including the salts thereof, with R5=Cl and R7=NHCH₂CH(C₆H₄OMe)(CH₂)₂NEt₂ and according to the following structure: 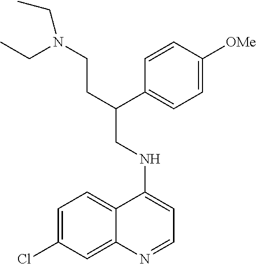 [SMILES: CCN(CC)CCC(CNc1ccnc2cc(Cl)ccc12)c1ccc(OC)cc1]
and/or the inhibitor for the nucleic-acid-detecting toll-like receptor comprises at least one compound, including the salts thereof, with R5=OMe, R7=NHCHMe(CH₂)₃NEt₂, R1=C₆H₅ and according to the following structure:

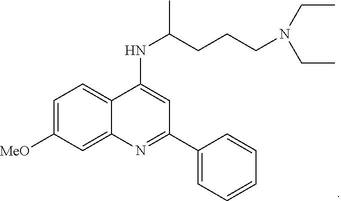 [SMILES: CCN(CC)CCCC(C)Nc1cc(-c2ccccc2)nc2cc(OC)ccc12]

15. Composition according to claim 9, wherein the inhibitor for the nucleic-acid-detecting toll-like receptor comprises at least one compound from the group of 9-aminoacridine, including the salts thereof, which has the following base structure: 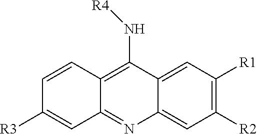
where R1 to R4 may be selected as desired.

16. Composition according to claim 15, wherein the inhibitor for the nucleic-acid-detecting toll-like receptor comprises at least quinacrine, including the salts thereof, with R1=OMe, R2=H, R3=Cl, R4=NHCHMe(CH₂)₃NEt₂ and according to the following structure:

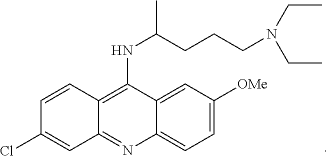 [SMILES: CCN(CC)CCCC(C)Nc1c2ccc(Cl)cc2nc2ccc(OC)cc12]

17. Composition according to claim 16, wherein the inhibitor for IKKe and/or TBK1 comprises one or more of the group of
BX795 (N-(3-((5-iodo-4-((3-(2-thienylcarbonyl) amino) propyl) amino)-2-pyrimidinyl) amino) phenyl)-1-pyrrolidinecarboxamide, CAS 702675-74-9);
BX320 (N-(3-((5-bromo-2-(3-(pyrrolidine-1-carbonylamino) anilino) pyrimidin-4-yl)amino) propyl)-2,2-dimethyl propanediamide, CAS 702676-93-5);
Cay10576 (5-(5,6-dimethoxybenzimidazol-1-yl)-3-(2-methanesulphonyl-benzyloxy)-thiophene-2-carbonitrile, CAS 862812-98-4);
Cay10575 (5-(5,6-dimethoxybenzimidazol-1-yl)-3-((4-methylsulphonyl) phenyl) methoxy)-2-thiophenecarboxamide, CAS 916985-21-2);
Amlexanox (2-amino-7-(1-methylethyl)-5-oxo-5H-[1]benzopyrano[2,3-b]pyridine-3-carboxylic acid, CAS 68302-57-8);
MRT-67307 (N-[3-[[5-cyclopropyl-2-[[3-(4-morpholinylmethyl)phenyl]amino]-4-pyrimidinyl]amino]propyl]-cyclobutanecarboxamide, CAS 1190378-57-4);
CYT387 (N-(cyanomethyl)-4-[2-[[4-(4-morpholinyl)phenyl]amino]-4-pyrimidinyl]-benzamide, CAS 1056634-68-4);
including the salts thereof.

18. Toolkit system for a transfection method according to claim 1, wherein the toolkit system has
at least one first inhibitor sub-composition, which comprises at least one inhibitor for IKKe and/or TBK1, and a second inhibitor sub-composition, which comprises at least one inhibitor for at least one nucleic-acid-detecting toll-like receptor,
or in that the toolkit system has
an inhibitor composition which comprises at least one inhibitor for IKKe and/or TBK1 and simultaneously at least one inhibitor for at least one nucleic-acid-detecting toll-like receptor.

19. Toolkit system according to claim 18, wherein the inhibitor composition or at least one of the inhibitor sub-compositions is a composition according to claim 11.

20. Toolkit system according to any of claims 18, wherein the toolkit system further comprises:
a gene delivery system composition comprising at least one non-viral gene delivery system, and/or
a nucleic acid composition comprising at least one modified or unmodified nucleic acid.

21. Toolkit system according to claim 18, wherein one or more of the compositions or sub-compositions forms a combined composition together with one or more other compositions or sub-compositions.

22. Transfection method according to claim 1, wherein, as an inhibitor for IKKe and/or TBK1, one or more of the group of
BX795 (N-(3-((5-iodo-4-((3-(2-thienylcarbonyl) amino) propyl) amino)-2-pyrimidinyl) amino) phenyl)-1-pyrrolidinecarboxamide, CAS 702675-74-9);
BX320 (N-(3-((5-bromo-2-(3-(pyrrolidine-1-carbonylamino) anilino) pyrimidin-4-yl)amino) propel)-2,2-dimethyl propanediamide, CAS 702676-93-5);
Cay10576 (5-(5,6-dimethoxybenzimidazol-1-yl)-3-(2-methanesulphonyl-benzyloxy)-thiophene-2-carbonitrile, CAS 862812-98-4);
Cay10575 (5-(5,6-dimethoxybenzimidazol-1-yl)-3-((4-methylsulphonyl) phenyl) methoxy)-2-thiophenecarboxamide, CAS 916985-21-2);
Amlexanox (2-amino-7-(1-methylethyl)-5-oxo-5H-[1]benzopyrano[2,3-b]pyridine-3-carboxylic acid, CAS 68302-57-8);
MRT-67307 (N-[3-[[5-cyclopropyl-2-[[3-(4-morpholinylmethyl)phenyl]amino]-4-pyrimidinyl]amino]propyl]-cyclobutanecarboxamide, CAS 1190378-57-4);
CYT387 (N-(cyanomethyl)-4-[2-[[4-(4-morpholinyl)phenyl]amino]-4-pyrimidinyl]-benzamide, CAS 1056634-68-4);
including the salts thereof, are selected.

23. Composition according to claim 11, wherein the inhibitor for IKKe and/or TBK1 comprises one of the group of
BX795 (N-(3-((5-iodo-4-((3-(2-thienylcarbonyl) amino) propyl) amino)-2-pyrimidinyl) amimo) phenyl)-1-pyrrolidinecarboxamide, CAS 702675-74-9);
BX320 (N-(3-((5-bromo-2-(3-(pyrrolidine-1-carbonylamino) anilino) pyrimidin-4-yl)amino) propyl)-2,2-dimethyl propanediamide, CAS 702676-93-5);
Cay10576 (5-(5,6-dimethoxybenzimidazol-1-yl)-3-(2-methanesulphonyl-benzyloxy)-thiophene-2-carbonitrile, CAS 862812-98-4);
Cay10575 (5-(5,6-dimethoxybenzimidazol-1-yl)-3-((4-methylsulphonyl) phenyl) methoxy)-2-thiophenecarboxamide, CAS 916985-21-2);
Amlexanox (2-amino-7-(1-methylethyl)-5-oxo-5H-[1]benzopyrano[2,3-b]pyridine-3-carboxylic acid, CAS 68302-57-8);
MRT-67307 (N-[3-[[5-cyclopropyl-2-[[3-(4-morpholinylmethyl)phenyl]amino]-4-pyrimidinyl]amino]propyl]-cyclobutanecarboxamide, CAS 1190378-57-4);
CYT387 (N-(cyanomethyl)-4-[2-[[4-(4-morpholinyl)phenyl]amino]-4-pyrimidinyl]-benzamide, CAS 1056634-68-4);
including the salts thereof.
